(19) United States
(12) Patent Application Publication
(10) Pub. No.: US 2015/0073567 A1
(43) Pub. Date: Mar. 12, 2015

(54) COMPOSITE PYLON FOR A PROSTHETIC DEVICE

(71) Applicant: Michael Thomas Wilson, Missouri City, TX (US)

(72) Inventor: Michael Thomas Wilson, Missouri City, TX (US)

(21) Appl. No.: 14/483,281

(22) Filed: Sep. 11, 2014

Related U.S. Application Data
(60) Provisional application No. 61/876,272, filed on Sep. 11, 2013.

(51) Int. Cl. A61F 2/78; A61F 2/80

(52) U.S. Cl. CPC A61F 2/7812; A61F 2/80
USPC 623/33; 623/38

(57) ABSTRACT

A pylon for a prosthetic device, the pylon having a central axis, a first end, and a second end. In an embodiment, the pylon includes a first connecting member disposed at the first end and configured to couple to a socket worn by an amputee. In addition, the pylon includes a second connecting member disposed at the second end and configured to couple to a prosthetic foot. Further, the pylon includes a pylon member extending axially from the first connecting member to the second connecting member, wherein the pylon member extends helically about the central axis.

### CROSS-REFERENCE TO RELATED APPLICATIONS

This application claims benefit of U.S. provisional patent application Ser. No. 61/876,272 filed Sep. 11, 2013, and entitled “Composite Pylon for a Prosthetic Device,” which is hereby incorporated herein by reference in its entirety.

### STATEMENT REGARDING FEDERALLY SPONSORED RESEARCH OR DEVELOPMENT

Not applicable.

### BACKGROUND

Embodiments disclosed herein relate generally to prosthetic limbs. More particularly, embodiments disclosed herein relate to flexible pylons for coupling prosthetic extremities to amputees.

Amputees typically employ the use of prosthetic limbs to replace the function of the particular limb that is missing. For patients who have had one of their legs amputated at or below the knee, the prosthesis typically includes a prosthetic foot, a socket within which the post-operative stump or residual limb is seated, and a rigid pylon extending between the socket and the prosthetic foot.

A useful prosthesis at least partially simulates the operation and motion of an anatomical foot. In addition, for Syme amputees (e.g., amputees who have sustained an ankle disarticulation), a useful prosthesis simulates the operation, flexion, and motion of an anatomical ankle An anatomical foot, including the ankle joint, is capable of motion around three perpendicular axes, as well as varying degrees of flexure. Specifically, the anatomical foot and ankle are capable of dorsiflexion, planiflexion, inversion, eversion, and transverse rotation. To achieve such functionality, some prosthetics include a distinct prosthetic ankle that is coupled to or incorporated into the prosthetic foot and that is capable of complicated motion (e.g., motion around two or three axes). However, inclusion of a prosthetic ankle may add bulk and additional weight to the prosthesis.

A useful prosthetic limb also provides a spring effect during use (e.g., be capable of absorbing, storing, and releasing energy). At a minimum, the prosthesis should store enough energy to return itself to a relaxed, unflexed position when external forces are removed. Such a spring effect may be accomplished in a conventional prosthetic foot by including various energy storing components such as coil springs. However, similar to prosthetic ankles, inclusion of such energy-storing components may significantly increase the weight of the prosthesis.

Although some conventional prosthetics are sufficiently strong and durable to withstand the stresses of repeated stepping motions over long periods of time, some conventional prostheses can become uncomfortable after extended periods of use due to excessive weight, bulkinesss, and inherent inflexibility of many of the components making up the prosthetic. Such discomfort may discourage use of the prosthetic and thus results in a reduced quality of life for the patient.

### BRIEF SUMMARY OF THE DISCLOSURE

Embodiments disclosed herein are directed to pylons for a prosthetic device. In an embodiment, the pylon has a central axis, a first end, and a second end. In addition, the pylon includes a first connecting member disposed at the first end and configured to couple to a socket worn by an amputee. Further, the pylon includes a second connecting member disposed at the second end and configured to couple to a prosthetic foot. Still further, the pylon includes a pylon member extending axially from the first connecting member to the second connecting member, wherein the pylon member extends helically about the central axis.

Embodiments disclosed herein are also directed to a prosthetic device. In an embodiment, the prosthetic device includes a socket configured to receive the residuum of an amputated limb. In addition, the prosthetic device includes a prosthetic extremity. Further, the prosthetic device includes a pylon extending between the socket and the prosthetic extremity, wherein the pylon has a central axis, a first end coupled to the socket, and a second end coupled to the prosthetic extremity. The pylon includes a first connecting member disposed at the first end and coupled to the socket, and a second connecting member disposed at the second end and coupled to the prosthetic extremity. In addition, the pylon includes a pylon member extending axially from the first connecting member to the second connecting member, wherein the pylon member extends helically about the central axis.

Embodiments disclosed herein are also directed to a pylon adapter for a prosthetic device. In an embodiment, the pylon adapter includes a central axis, a first end, and a second end. In addition, the pylon adapter includes a first coupling extending axially from the first end, the first coupling including a first receptacle that is defined by a frustoconical surface that is oriented at an angle α relative to the central axis, wherein the first receptacle is configured to receive a connecting member of a pylon of the prosthetic device. Further, the pylon adapter includes a second coupling extending axially from the second end, the second coupling including a second receptacle.

Embodiments described herein comprise a combination of features and advantages intended to address various shortcomings associated with certain prior devices, systems, and methods. The foregoing has outlined rather broadly the features and technical advantages of the invention in order that the detailed description of the invention that follows may be better understood. The various characteristics described above, as well as other features, will be readily apparent to those skilled in the art upon reading the following detailed description, and by referring to the accompanying drawings. It should be appreciated by those skilled in the art that the conception and the specific embodiments disclosed may be readily utilized as a basis for modifying or designing other structures for carrying out the same purposes. It should also be realized by those skilled in the art that such equivalent constructions do not depart from the spirit and scope of the disclosure.

### BRIEF DESCRIPTION OF THE DRAWINGS

For a detailed description of the preferred embodiments of the invention, reference will now be made to the accompanying drawings in which:

FIG. 1 is a schematic view of a conventional prosthetic for an amputee;

FIG. 2 is a schematic side view of an embodiment of a prosthetic including a helical pylon assembly in accordance with the principles disclosed herein;

FIG. 3 is a cross-sectional view of the helical pylon assembly of FIG. 2 taken along section III-III in FIG. 2;

FIG. 4 is a perspective side view of the helical pylon assembly of FIG. 2 illustrating only one of the pylon members;

FIG. 5 is an enlarged partial schematic side view of one of the pylon members of the helical pylon assembly of FIG. 2;

FIG. 6 is a schematic cross-sectional view of the lower pylon adapter of the helical pylon assembly of FIG. 2;

FIG. 7 is a schematic cross-sectional view of an alternative embodiment of a lower pylon adapter; and

FIG. 8 is a schematic partial schematic side view of an embodiment of a pylon member that can be used in the helical pylon assembly of FIG. 2.

### DETAILED DESCRIPTION OF THE PREFERRED EMBODIMENTS

The following discussion is directed to various exemplary embodiments. However, one skilled in the art will understand that the examples disclosed herein have broad application, and that the discussion of any embodiment is meant only to be exemplary of that embodiment, and not intended to suggest that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may not be shown in interest of clarity and conciseness.

In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection, or through an indirect connection via other devices, components, and connections. In addition, as used herein, the terms “axial” and “axially” generally mean along or parallel to a central axis (e.g., central axis of a body or a port), while the terms “radial” and “radially” generally mean perpendicular to the central axis. For instance, an axial distance refers to a distance measured along or parallel to the central axis, and a radial distance means a distance measured perpendicular to the central axis. As used herein, the phrase “omnidirectional flexibility” refers to flexibility in any direction. As used herein, the terms “approximately”, “substantially”, and “about” mean +/−10%.

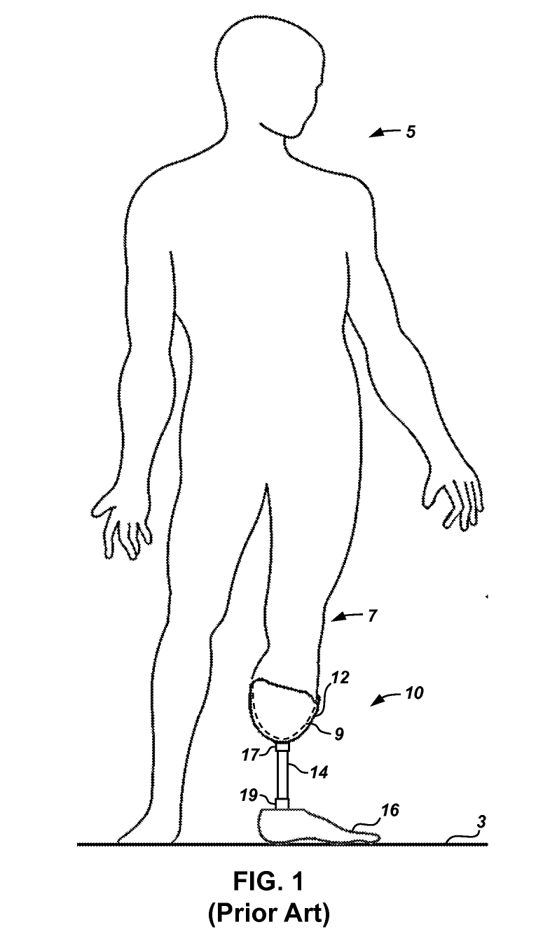

Referring now to FIG. 1, a schematic representation of a patient or user 5 utilizing a conventional prosthetic 10 is shown. In this embodiment, the leg 7 of user 5 has been amputated just below the knee such that leg 7 terminates in a stump or residuum 9. Prosthetic 10 includes a socket 12 at its upper end, a prosthetic foot 16 at its lower end, and a pylon 14 extending generally vertically between the socket 12 and foot 16. Socket 12 defines a receptacle that receives residuum 9. Pylon 14 is a rigid, straight column coupled to the socket 12 at one end and the foot 16 at the opposite end. In particular, pylon 14 is coupled to socket 12 with a first or upper socket adapter 17 and is coupled to foot 16 with a second or lower socket adapter 19. Typically, pylon 14 is a hollow tubular constructed from a rigid material such as, for example, a metal or a composite. Since pylon 14 is rigid and generally inflexible, as user 5 engages in dynamic motion (e.g., walking) with leg 7, reaction forces from the support surface 3 (e.g., ground or floor) are directed vertically through pylon 14, socket 12, and into residuum 9, thereby resulting in discomfort and a relatively limited movement potential for user 5. However, as will be described in more detail below, embodiments disclosed herein comprise helically shaped pylons that allow for enhanced omnidirectional flexibility during dynamic motion by a user (e.g., user 5), while still providing a strong, stable support column for transferring the weight of the user 5 into the support surface 3 during such use. The omnidirectional flexibility of the helically shaped pylons of the embodiments disclosed herein offers the potential to cushion at least a portion of the repetitive shocks transferred from the surface 3 to the residuum 9 of the user 5, as well as enhance overall flexibility of the prosthetic, thereby enhancing user comfort and maneuverability.

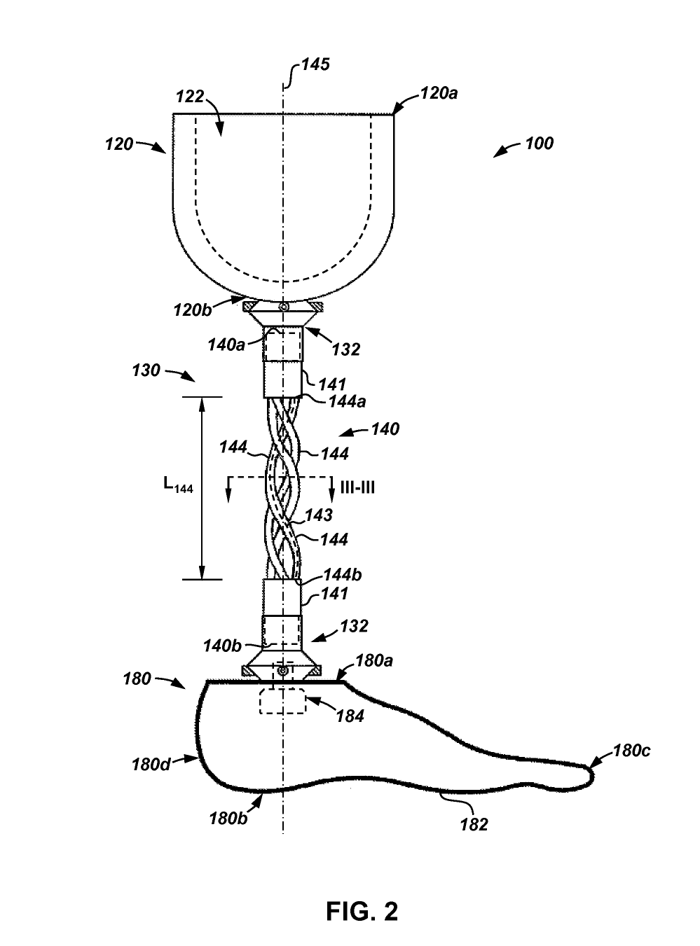

Referring now to FIG. 2, an embodiment of a prosthetic 100 in accordance with the principles disclosed herein is shown. Prosthetic 100 includes a socket 120, a pylon assembly 130, and a prosthetic foot 180. Socket 120 is substantially the same as socket 12 previously described, and in general, can comprise any suitable socket known in the art for use with a prosthetic limb. In this embodiment, socket 120 includes a first or upper end 120a, a second or lower end 120b opposite the upper end 120a, and a receptacle 122 extending axially from the upper end 120a. Receptacle 122 is configured to receive the post-operative stump or residual limb (e.g., residuum 9) of an amputated limb (e.g., leg 7) of a patient (e.g., user 5). As is known in the art, socket 120 can include various attachment members (e.g., buckles, snaps, hook and loop connectors) to ensure a secure connection to the user.

Prosthetic foot 180 is similar to prosthetic foot 16 previously described, and in general, can comprise any suitable prosthetic foot known in the art, such as, for example, any of the prosthetic feet disclosed in U.S. Pat. Nos. 8,118,879 and 7,871,443, each of which is incorporated herein by reference in its entirety for all purposes. As shown in FIG. 2, prosthetic foot 180 comprises a first or upper side 180a, a second or lower side 180b opposite the upper side 180a, a first or front end 180c, and a second or back end 180d opposite the front end 180c. An engagement surface 182 extends between the front end 180c and the back end 180d along the bottom side 180b. During use, at least a portion of surface 182 engages with a support surface (e.g., support surface 3) to provide support for a user using prosthetic 100. In addition, foot 180 includes a connector spindle 184 proximate the upper side 180a (note: spindle 184 is shown in FIG. 2 with a hidden line). Spindle 184 can comprise any suitable connector for a prosthetic foot, such as, for example, connectors manufactured by OTTO BOCH™ located in Minneapolis, Minn., or the like.

Referring still to FIG. 2, in this embodiment, pylon assembly 130 includes a pylon 140 and a pair of pylon adapters 132 coupled to opposite ends of pylon 140. One pylon adapter 132, also referred to herein as the “upper pylon adapter,” couples pylon 140 to socket 120, and the other pylon adapter 132, also referred to herein as the “lower pylon adapter,” couples pylon 140 to prosthetic foot 180.

Pylon 140 has a linear central axis 145, a first or upper end 140a, and a second or lower end 140b opposite end 140a. In addition, pylon 140 includes a pair of connecting members 141 and a plurality of circumferentially-spaced parallel pylon members 144. One connecting member 141 is disposed at upper end 140a, the other connecting member 141 is disposed at lower end 140b, and each pylon member 144 extends axially between connecting members 141. As will be described in more detail below, each pylon member 144 spirals helically around axis 145 as it extends between connecting members 141.

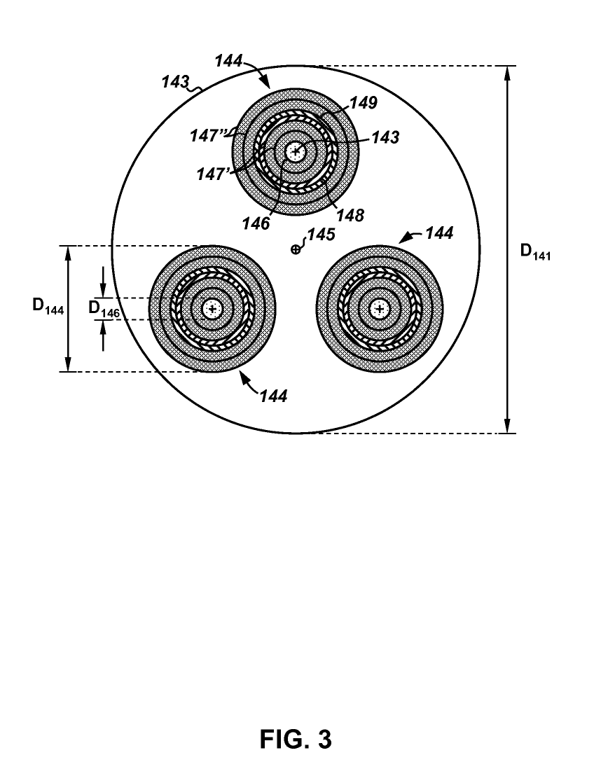

In this embodiment, each connecting member 141 is a cylindrical member sized and shaped to mate and engage with one of the adapters 132 of prosthetic 100. As will be described in more detail below, the member 141 disposed at upper end 140a is received within a mating receptacle in the upper adapter 132 and the member 141 disposed at lower end 140a is received within a mating receptacle in the lower adapter 132. As best shown in FIG. 3, in at least some embodiments, each connecting member 141 has a uniform outer diameter D₁₄₁ that is substantially the same as the diameter of the mating receptacles provided in the upper and lower adapters 132. Although each connecting member 141 has a cylindrical geometry in this embodiment, it should be appreciated that the specific geometry of each connecting member (e.g., each connecting member 141) can be varied, but is preferably sized and shaped to mate with the mating receptacles of the corresponding adapter (e.g., upper and lower adapters 132).

Each connecting member 141 is a rigid, solid structure designed to maintain the positions of pylon members 144 relative to each other while transferring loads between pylon members 144 and adapters 132. In this embodiment, each connecting member 141 is made from a composite material, and in particular, a carbon fiber and epoxy composite. However, in general, connecting members 141 can be made of any suitable material for supporting pylon members 144 and engaging with adapters 132 including, without limitation, metals and metal alloys (e.g., aluminum, steel, etc.), non-metals (e.g., resin, polymer, etc.), composite (e.g., carbon fiber composites), or combinations thereof.

Referring again to FIG. 2, each pylon member 144 has a central or longitudinal axis 143, a first or upper end 144a rigidly secured to connecting member 141 disposed at upper end 140a, and a second lower end 144b opposite the upper end 144a rigidly secured to connecting member 141 disposed at lower end 140b. In addition, each pylon member 144 has a length L₁₄₄ measured axially relative to axis 145 between the ends 144a, 144b. In this embodiment, each pylon member 144 has the same axial length L₁₄₄. In general, the axial lengths L₁₄₄ of the pylon members 144 will depend on a variety of factors including, without limitation, the height, weight, and walking style (e.g., gate) of the user (e.g., user 5), the location of the amputation, and the desired level of activity of the user. As best shown in FIG. 3, each pylon member 144 has an outer diameter D₁₄₄. In this embodiment, each pylon member 144 has the same outer diameter D₁₄₄ that is preferably between 0.25 in. and 0.625 in, and is more preferably between 0.37 in. and 0.625 in. In addition, in some embodiments, the axial length L₁₄₄ of each pylon member 144 is at least twelve (12) times the diameter D₁₄₄.

Referring again to FIG. 2, in this embodiment, pylon 140 includes three uniformly circumferentially-spaced pylon members 144. However, in other embodiments, more or less than three pylon members (e.g., pylon members 144) can be provided. For example, in one embodiment, the pylon (e.g., pylon 140) includes a total of four pylon members (e.g., four pylon members 144). In general, the number of pylon members is inversely related to the flexibility of the pylon. Thus, as the number of pylon members increases, the flexibility of the pylon decreases, and as the number of pylon members decreases, the flexibility of the pylon increases. However, it should be appreciated that the incremental decrease in flexibility also generally decreases with each additional pylon member 144 added to pylon 140 above a total of three pylon members 144. Thus, a determination as to the appropriate number of pylons members to include within the pylon is at least partially dictated by the desired flexibility of the pylon. It should also be appreciated that the resulting flexibility of pylon 140 is also greatly influenced by the rate of twist of the pylon members 144 (described in more detail below).

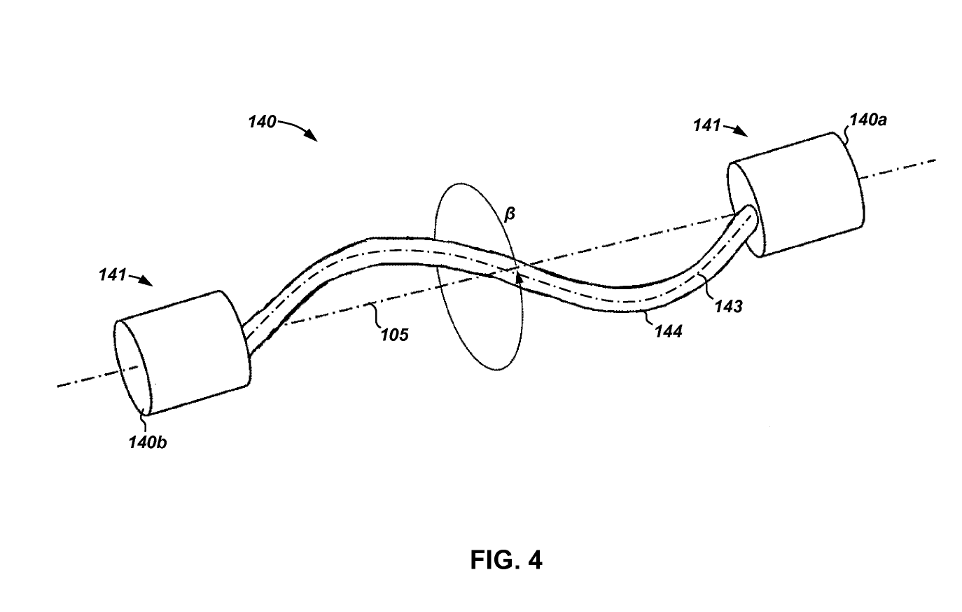

Referring now to FIG. 4, a single pylon member 144 is shown, it being understood that each pylon member 144 is the same. Pylon members 144 are uniformly circumferentially-spaced about axis 145 and oriented parallel to each other as shown in FIG. 3. In addition, each pylon member 144 spirals helically about axis 145 as it extends between connecting members 141. Accordingly, pylon members 144 may be described as “helical.” In this embodiment, although pylon members 144 are helical, the central axis 143 of each pylon member 144 is disposed at the same radius relative to axis 145. In other words, pylon members 144 of this embodiment do not incline to taper radially inward or radially outward moving towards either end 144a, 144b. Further, in some embodiments, the pylon members 144 are separated from the central axis 145 by a distance or radius that is equal to approximately 12.5% of the radius of each member 144 (i.e., 12.5% of one half of the diameter D₁₄₄), although other distances are possible. The helical geometry of each pylon member 144 can be described in terms of a helical angle β equal to the total angle, measured about axis 145, through which each member 144 extends between its ends 144a, 144b. As previously described, in this embodiment, pylon members 144 are parallel, and thus, the helical angle β for each pylon member 144 is the same. For embodiments described herein, the helical angle β for each pylon member 144 is preferably between 120° and 360° regardless of the axial length L₁₄₄ of each member 144. In the embodiment shown in FIG. 2, the helical angle β of each helical pylon member 144 is 360°, and thus, each helical pylon member 144 completes one full twist or turn about axis 145 between ends 144a, 144b. In general, the helical shape of each of the pylon members 144 provides omnidirectional flexibility for pylon 140 during use thereof. In other words, pylon 140 may flex such that the upper end 140a may flex or move in any direction relative to the lower end 140b and therefore foot 180. More particularly, without being limited to this or any other theory, in some embodiments, the helical shape of each member 144 allows each member 144 to wind or un-wind in response to an applied bending moment (e.g., such as would be applied during dynamic motion by a user), which thereby decreases the amount of compression and tension experienced by each member 144 during such deformation and thus increases the overall flexibility of pylon 140 along any direction relative to the axis 145. In addition, by maintaining a consistent helical angle β and number of pylon members 144, the flexibility of pylon 140 may be substantially held constant regardless of the axial length L₁₄₄.

Referring again to FIG. 2, in some embodiments the angular orientation of the ends 144a, 144b of members 144 within pylon 140, relative to the foot 180 and socket 120 is irrelevant. However, in other embodiments, such as, for example, those embodiments wherein a total of three pylon members 144 are included within pylon 140, it is preferable to angularly place the ends 144a, 144b of members 144 such that the lower end 144b of two of the members 144 faces (or is proximate) the front end 180c of foot 180 while the lower end 144b of one of the members 144 faces (or is proximate) the back end 180d of foot 180. Without being limited to this or any other theory, the above described angular arrangement of the pylon members 144 allows for increased level of vertical support for a user (e.g., user 5) toward the front end 180c of foot 180, which is desirable in at least some circumstances.

Referring now to FIG. 3, in this embodiment, each pylon member 144 includes several concentric annular layers. In particular, moving radially outward from the central axis 143, each member 144 includes a central core 146, an inner double layer of bi-directional carbon fiber 147′, a layer of infusion glass 148, a layer of bi-directional carbon glass 149, and an outer double layer of bi-directional carbon fiber 147″. Within each pylon member 144, the layers 146, 147′, 148, 149, 147″ are adhered or bonded together with a binding agent such as, for example, a resin (e.g., epoxy resin, a spray adhesive, etc.). Thus, within each pylon member 144, the layers 146, 147′, 148, 149, 147″ are joined together to form a single composite material capable of flexing as a single unit without the layers 146, 147′, 148, 149, 147″ delaminating or moving relative to each other.

In this embodiment, central core 146 is a solid rod made of urethane having a Durometer hardness of 70-90 Shore Scale A. In addition, in this embodiment, core 146 has a cylindrical geometry with an outer diameter D₁₄₆ preferably between 0.125 and 0.1875 in.

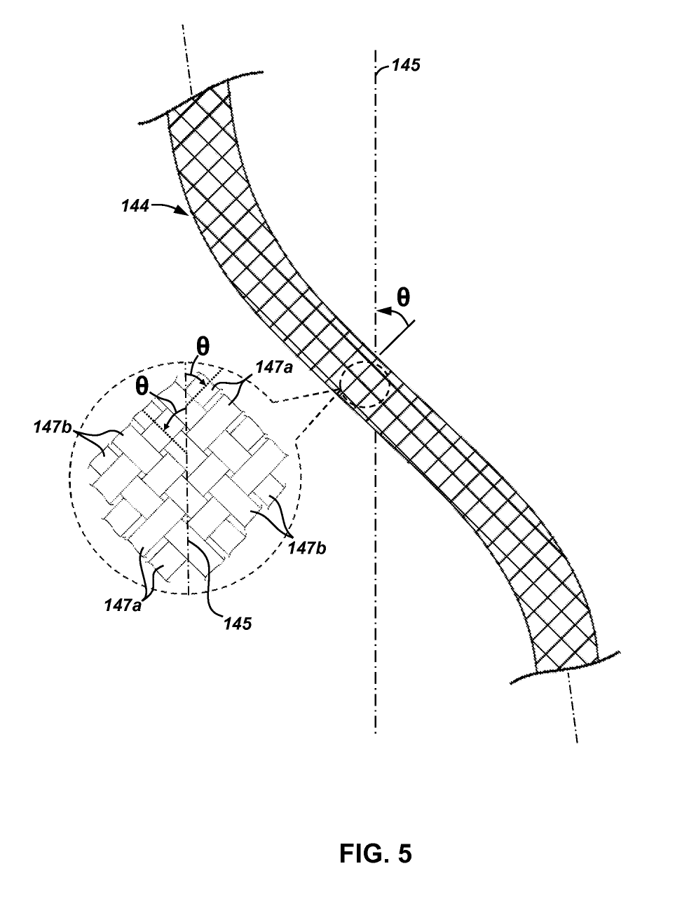

Referring now to FIG. 5, in some embodiments, each of the inner and outer layers of bi-directional carbon fiber 147′, 147″, respectively, comprises individual carbon fibers that are arranged along member 144 in an alternating fashion such that each fiber is oriented at an angle θ with respect to the central axis 145 of pylon 140. Each angle θ is preferably between 0° and 90°, more preferably between 30° and 60°, and even more preferably 45°. In this embodiment, each layer of bi-directional carbon fiber 147′, 147″ is oriented such that each angle θ is 45°. In particular, in the embodiment shown, each of the layers 147′, 147″ comprises a first plurality fibers 147a interwoven with a second plurality of fibers 147b. Each of the first plurality of fibers 147a is disposed at a 45° angle to the central axis 145 of the pylon 140. In addition, each of the second plurality of fibers 147b extends substantially perpendicular to each of the first plurality of fibers 147a such that each is also disposed at a 45° angle to the central axis 145 of the pylon 140. Similarly, in this embodiment, the layer of bi-directional carbon glass 149 comprises a plurality of individual fibers that are arranged in an alternating fashion such that each fiber is oriented at the same angle θ with respect to the central axis 145 of pylon 140. In other embodiments, the fibers of carbon glass within layer 149 may be oriented at a different angle θ than the fibers within the inner and outer layers of carbon fiber 147′, 147″, respectively, while still complying with the principles disclosed herein.

Referring again to FIG. 3, each layer of fusion glass 148 comprises a plurality of glass fibers oriented parallel to axis 143 of the corresponding pylon member 144. Additionally, glass 148 includes a plurality of slots or spaces between each of the glass fibers. The above described orientation and spaces allow for the ingress or flow of the binding agent (e.g., epoxy resin) axially between ends 144a, 144b of each member 144 as well as radially through the layer of fusion glass 148 during manufacturing. Further, it should be appreciated that pylon members 144 may be fabricated through any suitable method known in the art, such as, for example, resin transfer molding (“RTM”) or infusion molding, Prepreg autoclave molding, saturation molding, or some combination thereof.

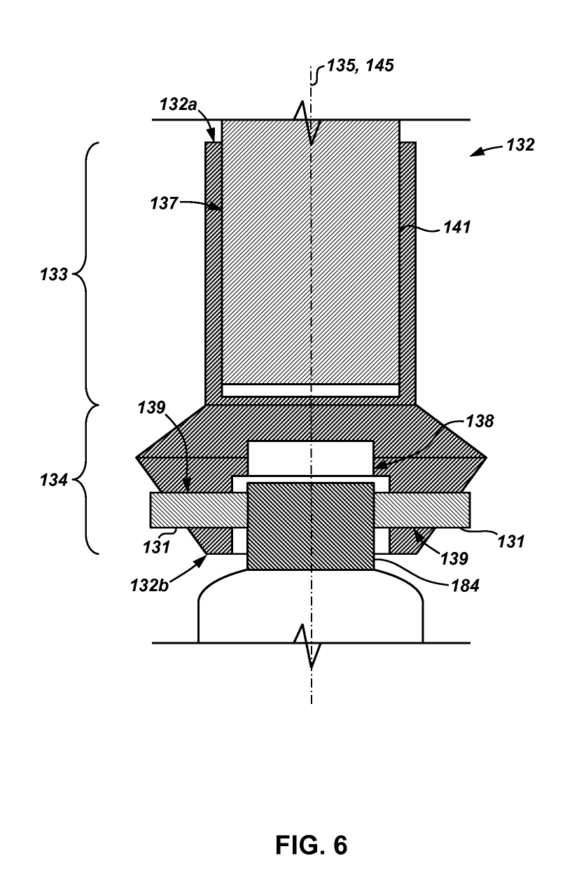

Referring now to FIG. 6, each pylon adapter 132 has a central axis 135, a first end 132a, a second end 132b opposite the end 132a, a first coupling 133 extending axially from the first end 132a, and a second coupling 134 extending axially from the second end 132b to coupling 133. First coupling 133 includes a receptacle 137 extending axially from the upper end 132a and second coupling 134 includes a receptacle 138 extending axially from the lower end 132b. As shown in FIG. 6, member 141 of pylon 140 is received and secured within receptacle 137, and spindle 184 is received and secured within receptacle 138 to connect foot 180 to pylon 140. Upper pylon adapter 132 is the same as lower pylon adapter 132 except that upper pylon adapter 132 is flipped or rotated approximately 180° such that connecting member 141 at upper end 140a of pylon 140 is received within receptacle 137, and a spindle 184 mounted to socket 120 is received and secured within receptacle 138 to connect socket 120 to pylon 140.

In general, each connecting member 141 can be secured within the corresponding receptacle 137 through any suitable means known in the art. For example, the connecting member 141 can be secured within receptacle 137 via an interference fit, an adjustable collar secured to adapter 132, a set screw extending radially into the receptacle 137 and engaging the radially outer surface of connecting member 141, a bonding agent, or combinations thereof. In this embodiment, receptacle 137 is defined by a cylindrical surface, however, in general, the receptacle 137 can have any suitable shape, but preferably has a shape that corresponds with the shape of the connecting member 141 disposed therein.

In this embodiment, each spindle 184 is secured within the corresponding receptacle 138 with a plurality of circumferentially-spaced set screws 131. Each set screw 131 is threadably disposed in one port 139 extending radially outward from receptacle 138 through coupling 134. Thus, screws 131 can be advanced radially into receptacle 138 and into engagement with spindle 184 by threadably advancing them through ports 139. In this embodiment, a total of four uniformly circumferentially-spaced ports 139 are provided. However, it should be appreciated that the number and arrangement of the ports 139 and set screws 131 may be varied while still complying with the principles disclosed herein.

Referring briefly to FIGS. 3 and 6, in some embodiments, it can be difficult to determine the appropriate size (e.g., diameter D₁₄₁) of the connecting member 141 in order to ensure a proper fit of member 141 within connector 132. In particular, the final or resulting diameter D₁₄₁ of the members 141 may fluctuate during the fabrication process due to properties of the material making up members 141 (e.g., epoxy or other resins, carbon fiber, etc.). For example, in some embodiments, the final value for the diameter D₁₄₁ may be reduced as much as 0.01 in from the initial diameter during the fabrication process. Thus, embodiments disclosed herein include alternative embodiments of pylon adapter 132 that include alternative geometries that both account for this fluctuation in the final sizing of the members 141 and ensure a more secure connection during use thereof.

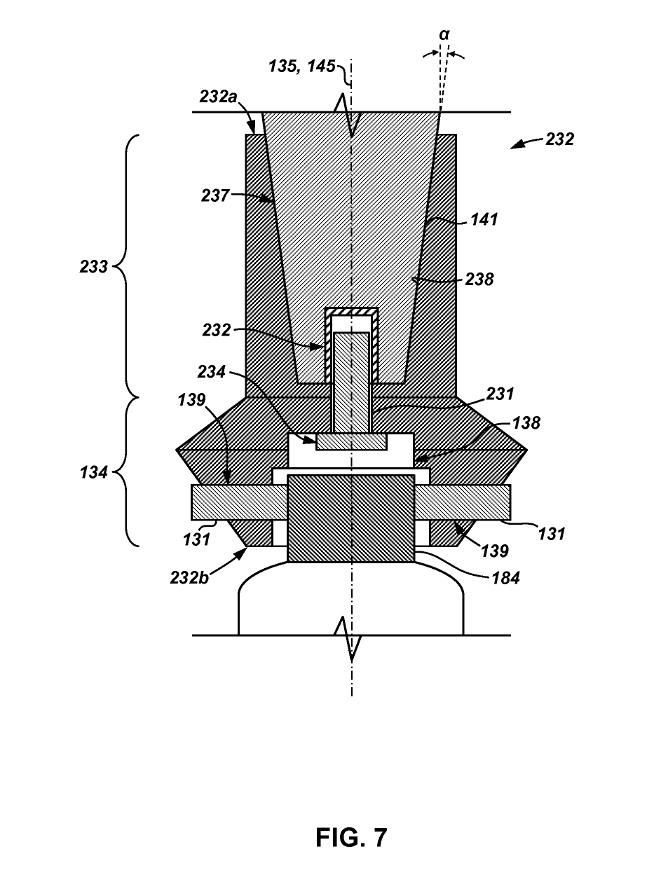

Referring now to FIG. 7, an alternative embodiment of pylon adapter 232 that can be used in place of either or both adapters 132 previously described is shown. Adapter 232 is substantially the same as adapter 132 previously described. In particular, lower pylon member 232 includes a first end 232a, a second end 232b opposite the first end 232a, a first coupling 233 extending axially from the first end 232a, and a second coupling 134, as previously described, extending from the second end 232b to coupling 233. First coupling 233 includes a receptacle 237 extending from first end 232a. Unlike receptacle 137 previously described, which is defined by a cylindrical surface, in this embodiment, receptacle 237 is defined by a frustoconical surface 238 that tapers slightly radially inward moving axially from first end 232a. In some embodiments, the frustoconical surface 238 of the receptacle 237 is disposed at an angle α relative to the central axis 135 that is preferably between 1-3°. In at least one particular embodiment, the frustoconical surface 238 defining the receptacle 237 is oriented such that there is approximately 1/32 inches of radial deflection (or radial run) for every 1⅛ inches of axial distance with respect to the axis 135. When a cylindrical connecting member 141 is axially advanced into the receptacle 237, an interference fit is formed between connecting member 141 and coupling 233. In some embodiments, connecting member 141 may also be frustoconical in shape in order to correspond with the shape of receptacle 237 during use. In at least some of these embodiments, the radially outermost surface of the connecting member 141 is formed to correspond to the radially inner surface 238 of the receptacle (i.e., the radially outermost surface of the member 141 slopes at the same angle α). Thus, regardless of whether the member 141 is cylindrical, frustoconical, or some other shape, a reduction in the nominal diameter of the members 141 is accounted for by simply seating the member 141 axially lower within the receptacle 237 (or axially closer to the coupling 134).

In addition, in this embodiment, a bore 231 extends axially between receptacles 138, 237. When connecting member 141 is seated in the receptacle 237, an internally threaded bore 232 in the lower end of connecting member 141 is coaxially aligned with the bore 231. Thereafter, a bolt 234 is axially advanced through bore 231 and threaded into bore 232, thereby pulling lower connecting member 141 axially downward within receptacle 237 and thus further ensuring a secure connection between the member 141 and receptacle 237.

Referring back to FIG. 2, during use a user (e.g., user 5) inserts the residuum (e.g., residuum 9) of an amputated leg (e.g., leg 7) into the socket 120 and secures it therein through any suitable method or device known in the art. Thereafter, the user may engage in some sort of dynamic motion such as, for example, walking During such activity reaction forces from a support surface (e.g., surface 3, or the ground) are transferred through the foot 180 and into the pylon 140 of pylon assembly 130. Because of the helical orientation of each of the pylon members 144 of pylon 140, pylon 140 bends and deforms omnidirectionally with respect to the axis 145 to at least partially dissipate such forces, thereby effectively increasing comfort for the user.

In the manner described, a user (e.g., user 5) may use a prosthetic (e.g., prosthetic 100) incorporating a helical pylon assembly (e.g., assembly 130) that allows for enhanced omnidirectional flexibility. Such enhanced flexibility may allow for increased comfort for users of such prosthetics and may thereby increase the overall quality of life for such amputee users.

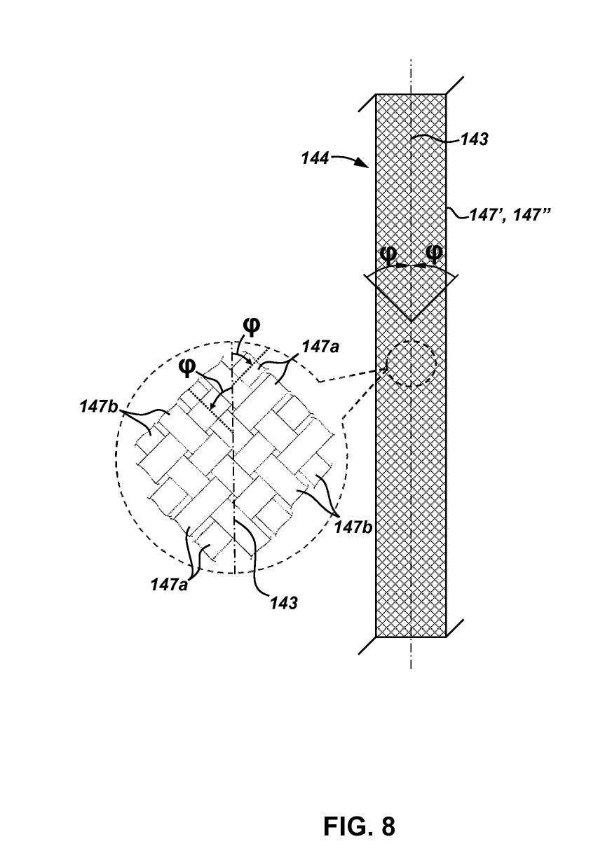

As previously described, in at least some embodiments, the individual carbon fibers that make up layers 147′, 147″ of bi-directional carbon fiber are oriented at the angle θ with respect to the central axis 145 of pylon 140. However, it should be appreciated that the individual carbon fibers of layers 147′, 147″ may have different orientations while still complying with the principles disclosed herein. For example, referring now to FIG. 8, a schematic representation of an alternative embodiment of a pylon member 244 is shown. Pylon members 244 can be employed in pylon assembly 130 in place of pylon members 144 previously described.

Referring still to FIG. 8, pylon member 244 is the same as pylon member 144 previously described except that the inner and outer layers of bi-directional carbon fiber 147′, 147″ are oriented in a different manner. More specifically, each of the inner and outer layers of bi-directional carbon fiber 147′, 147″, respectively, comprises individual carbon fibers that are arranged along member 244 in an alternating fashion such that each fiber is oriented at an angle φ with respect to the central axis 143 of pylon member 244 rather than being oriented at the angle θ with respect to the central axis 145 of pylon 140. Each angle φ is preferably between 0° and 90°, more preferably between 30° and 60°, and even more preferably 45°. In this embodiment, each layer of bi-directional carbon fiber 147′, 147″ is oriented such that each angle φ is 45°. Like pylon member 144, previously described, each of the layers 147′, 147″ of pylon 244 comprises a first plurality of fibers 147a interwoven with a second plurality of fibers 147b. Each of the first plurality of fibers 147a is disposed at a 45° angle to the central axis 143 of the pylon member 244. In addition, each of the second plurality of fibers 147b extends substantially perpendicular to each of the first plurality of fibers 147a such that each is also disposed at a 45° angle to the central axis 143 of the pylon member 244. Similarly, in this embodiment, the layer of bi-directional carbon glass 149 of pylon member 244 (see e.g., FIG. 3) comprises a plurality of individual fibers that are arranged in an alternating fashion such that each fiber is oriented at the same angle φ with respect to the central axis 143 along each member 144. In other embodiments, the fibers of carbon glass within layer 149 may be oriented at a different angle φ than the fibers within the inner and outer layers of carbon fiber 147′, 147″, respectively, while still complying with the principles disclosed herein.

While pylon assembly 130 has been described as including both an upper and lower pylon adapter 132, it should be appreciated that in other embodiments only one or neither of the pylon adapters 132 may be utilized while still complying with the principles disclosed herein. For example, the upper connecting member (e.g., upper connecting member 141) can be directly connected to a socket worn by the amputee (e.g., socket 120) and/or the lower connecting member (e.g., the lower connecting member 141) can be directly connected to a prosthetic foot (e.g., foot 180) through other suitable means. In addition, while the prosthetic 100 has been shown and described as a prosthesis for the lower leg, it should be appreciated that in other embodiments, prosthetic 100 may be configured to serve as any sort or type of lower limb prosthetic while still complying with the principles disclosed herein. Further, while the prosthetic 100 has been described as replacing an amputated limb, it should be appreciated that embodiments of the prosthetic 100 may also be utilized to replace limbs that were missing at birth. Still further, it should be appreciated that in some embodiments, one or both of the connecting members 141 of pylon 140 are formed in a mold that matches the shape of the matting receptacle of a corresponding pylon adapter (e.g., the receptacles 137, 237 of adapters 132, 232, respectively) to ensure a correct and secure fit between the member 141 and the corresponding adapter 132, 232 during use.

While preferred embodiments have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the invention. For example, the relative dimensions of various parts, the materials from which the various parts are made, and other parameters can be varied. Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps, but rather are used to simplify subsequent reference to such steps.

What is claimed is:

1. A pylon for a prosthetic device, the pylon having a central axis, a first end, and a second end, the pylon comprising:
a first connecting member disposed at the first end and configured to couple to a socket worn by an amputee;
a second connecting member disposed at the second end and configured to couple to a prosthetic foot; and
a pylon member extending axially from the first connecting member to the second connecting member, wherein the pylon member extends helically about the central axis.

2. The pylon of claim 1, wherein the pylon member extends through a helical angle β measured about the central axis, wherein the helical angle β is at least 120°.

3. The pylon of claim 2, wherein the helical angle β is less than or equal to 360°.

4. The pylon of claim 1, wherein the pylon member is made of a composite material including a plurality of concentrically arranged layers adhered to one another with a binding agent.

5. The pylon of claim 4, wherein at least one of the plurality of centrically arranged layers comprises a first layer of bi-directional carbon fiber.

6. The pylon of claim 5, wherein the first layer of bi-directional carbon fiber comprises a plurality of carbon fibers oriented approximately 45° to the central axis of the pylon.

7. The pylon of claim 4, wherein at least one of the plurality of concentrically arranged layers comprises a layer of infusion glass configured to allow the binding agent to flow axially therethrough.

8. The pylon of claim 1, further comprising a plurality of uniformly circumferentially-spaced parallel pylon members, wherein each pylon member extends axially from the first connecting member to the second connecting member, and wherein each pylon member extends helically about the central axis.

9. The pylon of claim 8, wherein each pylon member extends through a helical angle β measured about the central axis, wherein each helical angle β is greater than or equal to 180° and less than or equal to 360°.

10. The pylon of claim 1, wherein the first connecting member has a radially outer cylindrical or frustoconical surface.

11. A prosthetic device comprising:
a socket configured to receive the residuum of an amputated limb;
a prosthetic extremity;
a pylon extending between the socket and the prosthetic extremity, wherein the pylon has a central axis, a first end coupled to the socket, and a second end coupled to the prosthetic extremity;
wherein the pylon comprises:
a first connecting member disposed at the first end and coupled to the socket;
a second connecting member disposed at the second end and coupled to the prosthetic extremity; and
a pylon member extending axially from the first connecting member to the second connecting member, wherein the pylon member extends helically about the central axis.

12. The pylon of claim 11, wherein the pylon member extends through a helical angle β measured about the central axis, wherein the helical angle β is at least 120°.

13. The pylon of claim 12, wherein the helical angle β is less than or equal to 360°.

14. The pylon of claim 11, wherein the pylon member is made of a composite material including a plurality of concentrically arranged layers adhered to one another with a binding agent.

15. The pylon of claim 13, wherein at least one of the plurality of centrically arranged layers comprises a first layer of bi-directional carbon fiber.

16. The pylon of claim 15, wherein the first layer of bi-directional carbon fiber comprises a plurality of carbon fibers oriented approximately 45° to the central axis of the pylon.

17. The pylon of claim 16, wherein at least one of the plurality of concentrically arranged layers comprises a layer of infusion glass configured to allow the binding agent to flow axially therethrough.

18. The pylon of claim 11, further comprising a plurality of uniformly circumferentially-spaced parallel pylon members, wherein each pylon member extends axially from the first connecting member to the second connecting member, and wherein each pylon member extends helically about the central axis.

19. The pylon of claim 11, further comprising at least three circumferentially-spaced pylon members, wherein each pylon member extends axially from the first connecting member to the second connecting member, and wherein each pylon member extends helically about the central axis.

20. The pylon of claim 19, wherein the pylon members are uniformly circumferentially-spaced.

21. The pylon assembly of claim 20, wherein each pylon member extends through a helical angle β measured about the central axis, wherein each helical angle β is greater than or equal to 120° and less than or equal to 360°.

22. The prosthetic of claim 11, further comprising a first pylon adapter coupling the prosthetic extremity to the second connecting member, wherein the first pylon adapter includes:
a first receptacle that receives the second connecting member; and
a second receptacle that receives a connecting spindle of the prosthetic extremity.

23. The prosthetic of claim 22, wherein the first receptacle is defined by a frustoconical surface.

24. The prosthetic of claim 22, wherein the first pylon adapter has a first bore extending axially from the first receptacle and the second receptacle;
wherein the second connecting member has a second bore extending axially from the second end; and
wherein the first bore and the second bore are coaxially aligned;
wherein a bolt extends through the first bore and is threaded into the second bore.

25. A pylon adapter for a prosthetic device, comprising:
a central axis, a first end, and a second end;
a first coupling extending axially from the first end, the first coupling including a first receptacle that is defined by a frustoconical surface that is oriented at an angle α relative to the central axis, wherein the first receptacle is configured to receive a connecting member of a pylon of the prosthetic device; and
a second coupling extending axially from the second end, the second coupling including a second receptacle.

26. The pylon adapter of claim 25, wherein the angle α is between 1 and 3°.

27. The pylon adapter of claim 25, further comprising a bore extending axially between the first receptacle and the second receptacle, wherein the bore includes internal threads that are configured to mesh with external threads disposed along a connecting member.

28. The pylon adapter of claim 27, wherein the connecting member is a bolt.
